(19) United States
(12) Patent Application Publication
(10) Pub. No.: US 2011/0252488 A1
(43) Pub. Date: Oct. 13, 2011

(54) Method of Identifying Genes which Promote Hybrid Vigour and Hybrid Debility and Uses Thereof

(76) Inventor: Peter Hamilton Kay, Frankland (AU)

(21) Appl. No.: 10/588,577

(22) PCT Filed: Feb. 3, 2005
(86) PCT No.: PCT/AU05/00141

§ 371 Date: Jul. 28, 2008

(30) Foreign Application Priority Data
Feb. 3, 2004 (AU) 2004900471

(51) Int. Cl. A01K 67/027; C07H 21/04; A01K 67/00; C12Q 1/68; A01H 1/06

(52) U.S. Cl. 800/13; 435/6.12; 800/278; 800/25; 536/23.1

(57) ABSTRACT

The invention relates to a method of identifying candidate genes that are potentially useful in the diagnosis and treatment of disease and/or inducement of hybrid vigour. In particular, the present invention relates to a method for identifying candidate genes capable of producing hybrid vigour or hybrid debility in an animal or plant, comprising the steps of: (i) comparing the nucleotide sequence of alleles of candidate genes; (ii) identifying nucleotide sequence differences in the alleles from said animal or plant; and (iii) identifying that the amino acid sequence variation between alleles of the candidate gene are located within two or more different exons within the candidate gene.

### FIELD OF THE INVENTION

The invention relates to a method of identifying candidate genes that are potentially useful in the diagnosis and treatment of disease and/or inducement of hybrid vigour. The invention further relates to the use of hybrid mRNA molecules produced in vivo to overcome disease in a plant or animal and/or fix the heritability of hybrid vigour or other biologically advantageous or disadvantageous phenotype in a plant or animal.

### BACKGROUND OF THE INVENTION

It has been recognised for hundreds of years that some genetic factors which have the capacity to influence growth, viability or robustness of plants, animals and other organisms are more influential in offspring of genetically unrelated biologically normal parents. This biological phenomenon is referred to as hybrid vigour (HV) or heterosis. Offspring of genetically non-identical parents are referred to as heterozygous organisms.

Many models have been proposed to explain HV. In recent times a model akin to electric circuitry (Milborrow, J. Exp. Bot. 49, 1063 (1998) and another mathematically based model (Gordon, Heredity 83, 757 (1999)) have been put forward. However, as stated recently by Birchler, et al., The Plant Cell, 15, 2236 (2003), the underlying mechanism remains unknown. In 2003, Birchler and colleagues also predicted that “an eventual molecular explanation of heterosis will determine whether it can be manipulated for the benefit of agriculture and biotechnology.”

Although many theories have been advanced to explain this phenomenon, of major commercial importance, the view has long been held that HV is driven by novel proteins (such as hormones, enzymes or growth factors) synthesised uniquely in heterozygous or hybrid organisms compared to either parent (Schwartz, Proc. Natl. Acad. Sci. U.S.A. 46, 1210 (1960); Gill, in Genetics and Wheat Improvement, A. K. Gupta, Ed. (Oxford and IBH Publishing Co. New Delhi, 1977) pp 204-207) Scandalios et al. Arch. Biochem. Biophys. 153, 695 (1972), as found for example in maize hybrids (Romagnoli et al., Theor. Appl. Genet. 80, 769 (1990); Cheng et al., Chinese Sci. Bull. 41, 40 (1996)).

A further well known form of HV, in which an almost normal biological phenotype is restored in offspring of biologically defective parents, each of which carry different mutational forms of the same gene is referred to as intragenic or interallelic complementation (IC).

Some of the novel proteins formed in heterozygous offspring may, in contrast to HV, reduce the robustness, viability or well-being of or cause disease in an offspring compared to either parent. We refer to these biological phenotypes as hybrid debility (HD©).

Because the ability to synthesise novel proteins in heterozygous organisms does not conform to a Mendelian mode of inheritance, the heritability of HV has been impossible to predict reliably.

According to the central dogma, proteins are synthesised under the instruction of the inherited DNA sequence of genes through a series of well known biochemical steps. These steps comprise gene transcription, in which a primary RNA molecule (including intronic derived structures) is read off from the DNA sequence of genes inherited from each parent. Intron derived structures within the primary RNA molecule are spliced out by a large nucleoprotein complex called the spliceosome leaving only the exon-derived regions. The remaining exon-derived regions are then fused to form a mature messenger RNA (mRNA) molecule. The mRNA molecule then finally instructs the formation and amino acid sequence of a primary polypeptide by a process called translation. The Central dogma also states that following gene transcription the spliceosome engages a primary RNA molecule and removes its intron-derived elements by a mono-molecular threading or linear scanning process. Thus, exon-derived elements from the same primary RNA molecule are thought to be assembled to form mRNA in an orderly and well defined series of cis-reactions (Aebi et al. Trends Genet. 3, 102 (1987); Brown et al., Antonie van Leeuwenhoek 62, 35 (1992)).

The present invention provides basis for refuting this central dogma and thereby provide basis for artificially creating hybrid vigour and/or hybrid debility.

### SUMMARY OF THE INVENTION

In order to synthesise novel polypeptides or proteins, inventor has postulated that during the process of splicing, hybrid mRNA molecules could be generated in vivo by a primary RNA splicing mechanism that allows ligation of the 3′ end of an exon from a gene inherited from one parent with the 5′ end of the next downstream exon from the same gene from the other parent. Thus, inventor shows that novel or hybrid mRNA molecules, from which novel hybrid proteins can be synthesized, are generated by a previously unrecognised biochemical pathway which incorporates exons or parts thereof from each of the two different parental alleles into the same mature mRNA molecule. As detailed below, inventor has proven the existence of a biological pathway that assembles the said hybrid mRNA constructs.

Consequently, in the broadest aspect of the present invention, there is described a molecular genetic mechanism that explains how the biological phenotypes of HV and HD are generated in heterozygous offspring. The novel genetic structures formed in heterozygous offspring and which generate HV and HD are not obvious by reference to the heritable genetic code of their parents.

Accordingly, the invention provides a method of identifying candidate genes that are potentially useful in the diagnosis and treatment of disease and/or fixing the heritability of HV or other biologically advantageous or disadvantageous phenotype.

A first aspect provides a method for identifying candidate genes capable of producing hybrid vigour in an animal or plant, comprising the steps of:
(i) comparing the nucleotide sequence of alleles of candidate genes isolated from an animal or plant which exhibits hybrid vigour with the nucleotide sequences from the corresponding alleles isolated from the parents of said animal or plant;
(ii) identifying nucleotide sequence differences in the alleles from said animal or plant which exhibits hybrid vigour which codes for amino acid sequence variation; and
(iii) identifying that the amino acid sequence variation between alleles of the candidate gene in said animal or plant is encoded by nucleotide sequences which are located within two or more different exons within the candidate gene.

A second aspect provides a method for identifying candidate genes capable of producing hybrid debility (HD) in an animal or plant, comprising the steps of:
(i) comparing the nucleotide sequence of alleles of candidate genes isolated from an animal or plant which exhibits said hybrid debility (HD) with the nucleotide sequences from the corresponding alleles isolated from the parents of said animal or plant;
(ii) identifying nucleotide sequence differences in the alleles from said animal or plant which exhibits said hybrid debility (HD) which codes for amino acid sequence variation; and
(iii) identifying that the amino acid sequence variation between alleles of the candidate gene in said animal or plant is encoded by nucleotide sequences which are located within two or more different exons within the candidate gene.

A third aspect provides a method for producing hybrid vigour or hybrid debility in an animal or plant, comprising the steps of:
(i) comparing the nucleotide sequence of alleles isolated from a gene from an animal or plant which promotes hybrid vigour or hybrid debility with the nucleotide sequences from the corresponding alleles isolated from the parents of said animal or plant;
(ii) identifying nucleotide sequence differences in the alleles from said animal or plant which promote hybrid vigour or hybrid debility which code for amino acid sequence variation; and
(iii) identifying that the amino acid sequence variation between alleles of the candidate gene in said animal or plant is encoded by nucleotide sequences which are located within two or more different exons within the candidate gene.
(iv) preparing a construct comprising nucleotide sequence from the alleles which promotes hybrid vigour or hybrid debility within said animal or plant;
(v) transforming said construct into a recipient plant or animal cell;
(vi) regenerating a plant or animal, which expresses said construct, from said cell.

A fourth aspect provides a method of detecting the presence or absence of hybrid mRNA in a plant or animal comprising the step of isolating mRNA from a plant or animal and comparing the nucleotide sequence of said mRNA to the corresponding coding sequence of the plant or animal's alleles.

A fifth aspect provides a construct comprising a synthetic gene comprising exons from different alleles of a gene, wherein said alleles code for amino acid sequence variation wherein the variation occurs between different alleles.

The present invention further relates to the use of hybrid mRNA molecules produced in vivo to overcome disease in a plant or animal and/or induce hybrid vigour in a plant or animal.

The plant cell may be isolated from any higher plant, including gymnosperms, monocotyledonous and dicotyledenous plants, although all agriculturally important plant species are preferred. Preferably, the plant cell is isolated from a plant selected from the group consisting of barley, rye, sorghum, maize, soybean, wheat, corn, potato, cotton, rice, oilseed rape (including canola), sunflower, alfalfa, sugarcane, banana, blackberry, blueberry, strawberry, and raspberry, cantaloupe, carrot, cauliflower, coffee, cucumber, eggplant, grapes, honeydew, lettuce, mango, melon, onion, papaya, peas, peppers, pineapple, spinach, squash, sweet corn, tobacco, tomato, watermelon, rosaceous fruits (such as apple, peach, pear, cherry and plum) and vegetable brassicas (such as broccoli, cabbage, cauliflower, brussel sprouts and kohlrabi). Other crops, fruits and vegetables whose phenotype may be changed include barley, currant, avocado, citrus fruits such as oranges, lemons, grapefruit and tangerines, artichoke, cherries, nuts such as the walnut and peanut, endive, leek, roots, such as arrowroot, beet, cassava, turnip, radish, yam, sweet potato and beans. Other plant cells that might used in the present invention include cells isolated from woody species, such pine, poplar and eucalyptus. More preferably, the plant cell is a rice cell, a wheat cell, a barley cell, a rye cell, a sorghum cell or a maize cell.

The step of transforming the plant cell comprises any method known in the art, which is capable of stably transforming the plant cell. Suitable protocols are available for Leguminosae (alfalfa, soybean, clover, etc.), Umbelliferae (carrot, celery, parsnip), Cruciferae (cabbage, radish, rapeseed, broccoli, etc.), Curcurbitaceae (melons and cucumber), Gramineae (wheat, corn, rice, barley, millet, etc.), Solanaceae (potato, tomato, tobacco, peppers, etc.), and various other crops. See protocols described in Ammirato et al. (1984) Handbook of Plant Cell Culture—Crop Species. Macmillan Publ. Co. Shimamoto et al. (1989) Nature 338:274-276; Fromm et al. (1990) Bio/Technology 8:833-839; and Vasil et al. (1990) Bio/Technology 8:429-434.

Preferably, the plant cell is transformed by a method selected from the group consisting of homologous recombination microprojectile bombardment, PEG mediated transformation of protoplasts, electroporation, silicon carbide fibre mediated transformation, or Agrobacterium-mediated transformation. In a preferred embodiment of the invention the step of transforming comprises microprojectile bombardment by coating microprojectiles with DNA comprising the construct and contacting the recipient cells with the microprojectiles.

In another aspect, the invention provides a method of producing progeny comprising the steps of (a) preparing a plant according to the methods described above; and (b) crossing the plant with a second plant or with itself.

In yet another aspect, the invention provides a method of plant breeding comprising the steps of: (a) obtaining a progeny plant of any generation of a plant prepared according to the methods described above, wherein the progeny plant comprises said construct; and (b) crossing the plant with itself or a second plant.

In a sixth aspect the invention provides a method for producing genetically engineered or transgenic animal by inserting a synthetic gene into a somatic cell or cell nucleus prior to transferring the somatic cell or cell nucleus, wherein said synthetic gene comprises exons from different alleles of a gene, wherein said alleles code for amino acid sequence variation, wherein the variation does not occur in the same allele.

The invention further provides genetically engineered or transgenic animal obtained by the method of the sixth aspect.

The animal cells can be isolated from any animal although it is particularly useful for mammals and fish. Suitable mammalian sources include members of the Orders Primates, Rodentia, Lagomorpha, Cetacea, Carnivora, Perissodactyla and Artiodactyla. Members of the Orders Perissodactyla and Artiodactyla are particularly preferred because of their similar biology and economic importance.

For example, Artiodactyla comprise approximately 150 living species distributed through nine families: pigs (Suidae), peccaries (Tayassuidae), hippopotamuses

(Hippopotamidae), camels (Camelidae), chevrotains (Tragulidae), giraffes and okapi (Giraffidae), deer (Cervidae), pronghorn (Antilocapridae), and cattle, sheep, goats and antelope (Bovidae). Many of these animals are used as feed animals in various countries. More importantly, with respect to the present invention, many of the economically important animals such as goats, sheep, cattle and pigs have very similar biology and share high degrees of genomic homology.

The Order Perissodactyla comprises horses and donkeys, which are both economically important and closely related. Indeed, it is well known that horses and donkeys interbreed.

In one embodiment, the animal cells will be obtained from an ungulate. Preferably, the ungulate is selected from the group consisting of domestic or wild representatives of bovids, ovids, cervids, suids, equids and camelids. Examples of such representatives are cows or bulls, bison, buffalo, sheep, big-horn sheep, horses, ponies, donkeys, mule, deer, elk, caribou, goat, water buffalo, camels, llama, alpaca, and pigs. Especially preferred in the bovine species are Bos taurus, Bos indicus, and Bos buffaloes cows or bulls.

In one embodiment the animal cells are isolated from aquatic organisms such as vertebrate and invertebrate marine animals. More preferably the aquatic organism is selected from the group consisting of fish, amphibians and molluscs. Fish include; but are not limited to, zebrafish, European carp, salmon, mosquito fish, tench, lampreys, round gobies, tilapia and trout. Amphibians include; but are not limited to, toads and frogs. Molluscs include; but are not limited to, Pacific oysters, zebra mussels, striped mussels, New Zealand screw shells, the Golden Apple Snail, the Giant African Snail, and the disease vectoring snails in the genera Biomphalaria and Bulinus.

Transformation of constructs of the invention into animal cells is preferably by homologous recombination. Homologous recombination (see Molecular Genetics, U. Melcher (1998) is preferably induced by addition of the engineered construct to cells such as embryonic stem cells grown in tissue culture which contain the targeted gene. More preferably, transformed stem cells are injected into a new blastocyst causing fixation of the new construct in the mature animal.

### DETAILED DESCRIPTION OF THE INVENTION

All publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

The practice of the present invention employs, unless otherwise indicated, conventional molecular biology, plant and animal biology, and recombinant DNA techniques within the skill of the art. Such techniques are well known to the skilled worker, and are explained fully in the literature. See, eg., Maniatis, Fritsch & Sambrook, “Molecular Cloning: A Laboratory Manual” (1982); “DNA Cloning: A Practical Approach,” Volumes I and II (D. N. Glover, Ed., 1985); “Oligonucleotide Synthesis” (M. J. Gait, Ed., 1984); “Nucleic Acid Hybridization” (B. D. Hames & S. J. Higgins, eds., 1985); “Transcription and Translation” (B. D. Hames & S. J. Higgins, eds., 1984); B. Perbal, “A Practical Guide to Molecular Cloning” (1984), and Sambrook, et al., “Molecular Cloning: a Laboratory Manual” 12^{th} edition (1989).

The description that follows makes use of a number of terms used in recombinant DNA technology. Unless defined otherwise, all technical and scientific terms used herein have the meaning commonly understood by a person skilled in the art to which this invention belongs. The following references provide one of skill with a general definition of many of the terms used in this invention: Singleton, et al., “Dictionary of Microbiology and Molecular Biology” (2nd ed. 1994); “The Cambridge Dictionary of Science and Technology” (Walker ed., 1988); “The Glossary of Genetics” 5^{th} Ed., _{Rieger}, _{R}., et al. (eds.), Springer Verlag (1991); and Hale & Marham, “The Harper Collins Dictionary of Biology” (1991). Generally, the nomenclature and the laboratory procedures in plant and animal maintenance and breeding as well as recombinant DNA technology described herein are those well known and commonly employed in the art.

It is understood that the invention is not limited to the particular materials and methods described, as these may vary. It is also to be understood that the terminology used herein is for the purpose of describing particular embodiments only, and it is not intended to limit the scope of the present invention which will be limited only by the appended claims. It must be noted that as used herein and in the appended claims, the singular forms “a”, “an”, and “the” include plural reference unless the context clearly dictates otherwise. Thus, for example, a reference to “a plant cell” includes a plurality of such plant cells, and a reference to “an animal cell” is a reference to one or more animal cells. Although any materials and methods similar or equivalent to those described herein can be used to practice or test the present invention, the preferred materials and methods are now described.

### DEFINITIONS

The terms “polynucleotide”, “polynucleotide sequence”, “nucleic acid sequence”, and “nucleic acid fragment”/“isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof

The term “isolated” polynucleotide refers to a polynucleotide that is substantially free from other nucleic acid sequences, such as and not limited to other chromosomal and extrachromosomal DNA and RNA that normally accompany or interact with the isolated polynucleotide as found in its naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesised polynucleotides.

The term “recombinant” means, for example, that a nucleic acid sequence is made by an artificial combination of two otherwise separated segments of sequence, eg., by chemical synthesis or by the manipulation of isolated nucleic acids by genetic engineering techniques.

As used herein, “substantially similar” refers to nucleic acid molecules wherein changes in one or more nucleotide bases either results in no change to the amino acid sequence coded or substitution of one or more amino acids does not affect the functional properties of the polypeptide encoded by the nucleotide sequence.

Substantially similar nucleic acid molecules may also be characterised by their ability to hybridise. Estimates of such homology are provided by either DNA-DNA or DNA-RNA hybridisation under conditions of stringency as is well understood by those skilled in the art (Hames and Higgins, Eds. (1985) Nucleic Acid Hybridisation, IRL Press, Oxford, U.K.). Post-hybridisation washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C.

“Synthetic gene” or “synthetic nucleic acid fragments” can be assembled from oligonucleotide building blocks that are chemically synthesised using procedures known to those skilled in the art. These building blocks are ligated and annealed to form larger nucleic acid molecules, which may then be enzymatically assembled to construct the entire desired nucleic acid molecule. “Chemically synthesised”, as related to a nucleic acid fragment, means that the component nucleotides were assembled in vitro. Manual chemical synthesis of nucleic acid fragments may be accomplished using well established procedures, or automated chemical synthesis can be performed using one of a number of commercially available machines. Accordingly, the nucleic acid fragments can be tailored for optimal gene expression based on optimisation of the nucleotide sequence to reflect the codon bias of the host cell. The skilled artisan appreciates the likelihood of successful gene expression if codon usage is biased towards those codons favoured by the host. Determination of preferred codons can be based on a survey of genes derived from the host cell where sequence information is available.

“Candidate gene allele” refers to normal alleles of the candidate gene sequence as well as alleles carrying variations that predispose individuals to develop hybrid vigour or hybrid debility. Accordingly, as used herein, the terms “candidate gene sequence,” and “candidate gene allele” refer to the double-stranded DNA comprising the gene sequence, allele, or region, as well as either of the single-stranded DNAs comprising the gene sequence, allele or region (i.e. either of the coding and non-coding strands).

“Candidate gene” or “gene” refers to a nucleic acid fragment that expresses a specific protein, including regulatory sequences preceding (5′ non-coding sequences), in between and following (3′ non-coding sequences) the coding sequence. “Native gene” refers to a gene as found in nature with its own regulatory sequences. “Chimeric gene” or “exogenous gene” are used herein interchangeably and refer to any gene that is not a native gene, comprising regulatory and coding sequences that are not found together in nature. Accordingly, a chimeric or exogenous gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign-gene” refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a nucleotide sequence that code for a specific amino acid sequence. The term “amino acid sequence homolog” or “identity” refers to a protein with a similar amino acid sequence. One of skill will realise that the critical amino acid sequence is within a functional domain of a protein. Thus, it may be possible for a homologous protein to have less than 40% homology over the length of the amino acid sequence, but greater than 90% homology in one functional domain.

Amino acids may be referred to herein by either their commonly known three letter symbols or by the one-letter symbols recommended by the IUPAC-IUB Biochemical Nomenclature Commission.

Nucleotides, likewise, may be referred to by their commonly accepted single-letter codes.

“Conservative modified variants” applies to both amino acid and nucleic acid sequences. With respect to particular nucleic acid sequences, conservatively modified variants refers to those nucleic acids that encode identical or essentially identical amino acid sequences, or where the nucleic acid does not encode an amino acid sequence, to essentially identical sequences.

Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein, which encodes a polypeptide, also describes every possible silent variation of the nucleic acid. One of skill will recognise that each codon in a nucleic acid (except AUG, which is ordinarily the only codon for methionine) can be modified to yield a functionally identical molecule. Accordingly, each silent variation of a nucleic acid, which encodes a polypeptide, is implicit in each described sequence.

As to amino acid sequences, one of skill will recognise that a “conservatively modified variant” will result from an alteration to the coding sequence which results in the substitution of an amino acid with a chemically similar amino acid. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W). (See, e.g., Creighton, Proteins (1984)).

The term “non-conservative modified variation” refers to amino acid variation that does not involve conservative substitution. For example, the substitution of an alanine for a phenylalanine would constitute a non-conservative modified variation.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences and sequences that affect splice site selection.

“Promoter” refers to a nucleotide sequence capable of controlling the expression of a coding sequence or functional RNA. In general, a coding sequence is located 3′ to a promoter sequence. The promoter sequence consists of proximal and more distal upstream elements, the latter elements often referred to as enhancers. Accordingly, an “enhancer” is a nucleotide sequence which can stimulate promoter activity and may be an innate element of the promoter or a heterologous element inserted to enhance the level or tissue-specificity of a promoter. Promoters may be derived in their entirety from a native gene, or may be composed of different elements derived from different promoters found in nature, or may even comprise synthetic nucleotide segments. It is understood by those skilled in the art that different promoters may direct the expression of a gene in different tissues or cell types, or at different stages of development, or in response to different environmental conditions. Promoters which cause a nucleic acid fragment to be expressed in most cell types at most times are commonly referred to as “constitutive promoters”.

“Translation leader sequence” refers to a nucleotide sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency.

Examples of translation leader sequences have been described (Turner and Foster (1995) Mol. Biotechnol. 3:225-236). “3′ non-coding sequences” refer to nucleotide sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al. (1989) Plant Cell 1:671-680.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. “Messenger RNA (mRNA)” refers to the RNA that is without introns and that can be translated into polypeptides by the cell. “cDNA” refers to DNA that is complementary to and derived from an mRNA template. The cDNA can be single-stranded or converted to double stranded form using, for example, the Klenow fragment of DNA polymerase I. “Sense-RNA” refers to an RNA transcript that includes the mRNA and so can be translated into a polypeptide by the cell. “Functional RNA” refers to sense RNA, antisense RNA, ribozyme RNA, or other RNA that may not be translated but yet has an effect on cellular processes.

The term “operably linked” refers to the association of two or more nucleic acid fragments on a single polynucleotide so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) derived from the nucleic acid fragment of the invention. Expression may also refer to translation of mRNA into a polypeptide.

A “protein” or “polypeptide” is a chain of amino acids arranged in a specific order determined by the coding sequence in a polynucleotide encoding the polypeptide. Each protein or polypeptide has a unique function.

“Altered levels” or “altered expression” refers to the production of gene product(s) in transgenic organisms in amounts or proportions that differ from that of normal or non-transformed organisms.

“Mature protein” or the term “mature” when used in describing a protein refers to a post-translationally processed polypeptide; i.e., one from which any pre- or propeptides present in the primary translation product have been removed. “Precursor protein” or the term “precursor” when used in describing a protein refers to the primary product of translation of mRNA; i.e., with pre- and propeptides still present. Pre- and propeptides may be but are not limited to intracellular localization signals.

“Transformation” refers to the transfer of a nucleic acid fragment into the genome of host organism, resulting in genetically stable inheritance. Host organisms containing the transformed nucleic acid fragments are referred to as “transgenic” organisms. Examples of methods of plant transformation include Agrobacterium-mediated transformation (De Blaere et al. (1987) Meth. Enzymol. 143:277) homologous recombination and particle-accelerated or “gene gun” transformation technology (Klein et al. (1987) Nature (London) 327:70-73; U.S. Pat. No. 4,945,050, incorporated herein by reference). Thus, isolated polynucleotides of the present invention can be incorporated into recombinant constructs, typically DNA constructs, capable of introduction into and replication in a host cell. Such a construct can be a vector that includes a replication system and sequences that are capable of transcription and translation of a polypeptide-encoding sequence in a given host cell. A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Flevin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

“PCR” or “polymerase chain reaction” is well known by those skilled in the art as a technique used for the amplification of specific DNA segments (U.S. Pat. Nos. 4,683,195 and 4,800,159).

The term “progeny” refers to any subsequent generation, including the seeds and plants therefrom, which is derived from a particular parental plant or set of parental plants.

“Regeneration” is the process of growing a plant from a plant cell (eg., plant protoplast or explant).

“Selected DNA” is a segment of DNA which has been introduced into a host genome. Preferred selected DNAs will include one or more exogenous genes and the elements for expressing an exogenous gene in a host cell, for example, a promoter and a terminator. Benefit may be realised by including one or more enhancer elements with the selected DNA.

A “transformed cell” is a cell whose DNA has been altered by the introduction of an exogenous DNA molecule into that cell.

A “transgenic plant” is a plant or progeny of any subsequent generation derived therefrom, of a transformed plant cell or protoplast, wherein the plant DNA contains an introduced exogenous DNA molecule not originally present in a native, non-transgenic plant of the same strain. The transgenic plant may additionally contain sequences which are native to the plant being transformed, but wherein the “exogenous” gene has been altered by gene technological means in order to alter the structure of the original or wild-type gene product or the level or pattern of expression of the gene.

A “vector” is a DNA molecule capable of replication in a host cell and/or to which another DNA segment can be operatively linked so as to bring about replication of the attached segment. A plasmid is an exemplary vector or a DNA molecule used to carry new genes into cells. A plasmid is an exemplary vector which is an independent, stable, self-replicating piece of DNA

As used herein, the term “genotype” means the genetic makeup of an individual cell, cell culture, plant or animal.

As used herein, the term “heterozygote” means a diploid or polyploid individual cell or plant or animal having different alleles (forms of a given gene) at least at one locus.

As used herein,.the term “heterozygous” means the presence of different alleles (forms of a given gene) at a particular gene locus.

As used herein, the term “homozygote” means an individual cell or plant having the same alleles at one or more loci.

As used herein, the term “homozygous” means the presence of identical alleles at one or more loci in homologous chromosomal segments.

### EXPERIMENTAL PROTOCOLS

One aspect of the invention relates to a method of identifying candidate genes. The term “candidate gene” refers to a gene or genes that have the potential to produce hybrid vigour or hybrid debility. The terms “hybrid vigour” or “heterosis” are used herein interchangeably and means an increase in the performance of hybrids over that of purebreds, most noticeably in traits like growth rate, fertility and disease resistance. In particular the candidate genes are those genes, which are heterozygous and functional in one animal or plant and which lead to hybrid vigour. In one embodiment the alleles of each gene will comprise nucleotide sequence variation in different exons of the coding sequence wherein the variations lead to amino acid sequence variation. For example, if a plant or animal comprises two alleles of gene X ie heterozygote for gene X, it satisfies the first requirement as a candidate gene of the present invention. A second criterion would be sequence variation in the nucleotide sequence, which variation leads to amino acid sequence variation in any expressed protein. A third criterion would be that each allele contains sequence variation, wherein the sequence variation is not found at the same position in each allele. For example, in gene X supra each allele may contain a sequence variation wherein in allele 1 the variation is at position 1, while in allele 2 the variation is at position 20. This would satisfy the 2^{nd} and 3^{rd} criteria if the variation leads to a change in the amino acid sequence. A fourth criterion would be the variations appearing in different exons rather than merely in different positions within the same exon.

In one embodiment, the candidate gene would have one or more nucleotide sequence variations, which code for amino acid variations, wherein the variations are found in different exons on different alleles.

The candidate genes would preferably produce mRNA of at least three species: one mRNA molecule which is identical to one allele, one that was identical to the coding sequence of the second allele and hybrid mRNA molecules that comprise a combination of exons from both alleles. Obviously depending upon the number of exons the number of different species of hybrid mRNA molecules would be numerous.

Methods of identifying candidate genes would be relatively simple these could be identified by routine sequence analysis or Southern blot analysis of PCR amplified segments of candidate genes (see, for example, Gieselmann et al. (1989, Proc. Natl. Acad. Sci. U.S.A. 86:9436-9440). It will also be appreciated by those skilled in the art that detection of amplification in homogenous and/or closed tubes can be carried out using numerous means in the art, for example using TaqMan® hybridisation probes in the PCR reaction and measurement of fluorescence specific for the target nucleic acids once sufficient amplification has taken place. However, because of the nature and speed of the Roche Lightcycler®, the preferred method is by using real-time PCR and melting curve analysis on the Roche Lightcycler® using fluorescent labelled hybridisation oligonucleotides.

Although those skilled in the art will be aware that other similar quantitative “real-time” and homogenous nucleic acid amplification/detection systems exist such as those based on the TaqMan approach (U.S. Pat. Nos. 5,538,848 and 5,691,146), fluorescence polarisation assays (eg Gibson et al., Clin Chem, 1997; 43: 1336-1341), and the Invader assay (eg Agarwal P et al., Diagn Mol Pathol 2000 September; 9(3): 158-164; Ryan D et al, Mol Diagn 1999 June; 4(2): 135-144). Such systems would also be adaptable to use the invention described, enabling real-time monitoring of nucleic acid amplification and allele discrimination for detection of gene mutations and polymorphisms if appropriately designed.

Once a candidate gene has been identified it can then be isolated or produced synthetically to be used in other aspects of the invention. For example, a candidate gene, which is suspected of producing hybrid vigour, could be transformed into a host cell (plant or animal) and a transgenic plant or animal regenerated.

Methods for generating transgenic animal cells typically include the steps of (1) assembling a suitable DNA construct useful for inserting a specific DNA sequence into the nuclear genome of a cell; (2) transfecting the DNA construct into the cells; (3) allowing random insertion and/or homologous recombination to occur. The modification resulting from this process may be the insertion of a suitable DNA construct(s) into the target genome; deletion of DNA from the target genome; and/or mutation of the target genome.

DNA constructs can comprise a gene of interest, for example, a synthetic gene comprising exons taken from different alleles, wherein different exons comprise different sequences, as well as a variety of elements including regulatory promoters, insulators, enhancers, and repressors as well as elements for ribosomal binding to the RNA transcribed from the DNA construct. These examples are well known to a person of ordinary skill in the art and are not meant to be limiting.

Due to the effective recombinant DNA techniques available in conjunction with DNA sequences for regulatory elements and genes readily available in data bases and the commercial sector, a person of ordinary skill in the art can readily generate a DNA construct appropriate for establishing transgenic animal cells using the materials and methods described herein.

For example, if the entire nucleotide coding sequence for a candidate gene is not obtained in a single cDNA, genomic DNA, or other DNA, as determined, for example, by DNA sequencing or restriction endonuclease analysis, then appropriate DNA fragments (eg., restriction fragments or PCR amplification products) may be recovered from several DNAs and covalently joined to one another to construct the entire coding sequence. The preferred means of covalently joining DNA fragments is by ligation using a DNA ligase enzyme, such as T4 DNA ligase. The isolated candidate gene can then be incorporated into a plasmid or expression vector.

Transfection techniques for animal cells are well known to a person of ordinary skill in the art and materials and methods for carrying out transfection of DNA constructs into animal cells are commercially available. Materials typically used to transfect animal cells with DNA constructs are lipophilic compounds, such as Lipofectin™ for example. Particular lipophilic compounds can be induced to form liposomes for mediating transfection of the DNA construct into the cells.

Target sequences from the DNA construct can be inserted into specific regions of the nuclear genome by rational design of the DNA construct. These design techniques and methods are well known to a person of ordinary skill in the art. See, for example, U.S. Pat. No. 5,633,067; U.S. Pat. No. 5,612,205 and PCT publication WO93/22432, all of which are incorporated by reference herein in their entirety. Once the desired DNA sequence is inserted into the nuclear genome, the location of the insertion region as well as the frequency with which the desired DNA sequence has inserted into the nuclear genome can be identified by methods well known to those skilled in the art.

Once the transgene is inserted into the nuclear genome of a donor cell, that cell, like other donor cells of the invention, can be used as a nuclear donor in nuclear transfer methods. The means of transferring the nucleus of a cell into an oocyte preferably involves cell fusion to form a reconstituted cell.

Fusion is typically induced by application of a DC electrical pulse across the contact/fusion plane, but additional AC current may be used to assist alignment of donor and recipient cells. Electrofusion produces a pulse of electricity that is sufficient to cause a transient breakdown of the plasma membrane and which is short enough that the membrane reforms rapidly. Thus, if ₜwo adjacent membranes are induced to breakdown and upon reformation the lipid bilayers intermingle, small channels will open between the two cells. Due to the thermodynamic instability of such a small opening, it enlarges until the two cells become one. Reference is made to U.S. Pat. No. 4,997,384 by Prather et al., (incorporated by reference in its entirety herein) for a further discussion of this process. A variety of electrofusion media can be used including eg., sucrose, mannitol, sorbitol and phosphate buffered solution.

Fusion can also be accomplished using Sendai virus as a fusogenic agent (Graham, Wister Inot. Symp. Monogr., 9, 19, 1969). Fusion may also be induced by exposure of the cells to fusion-promoting chemicals, such as polyethylene glycol.

Preferably, the donor animal cell and oocyte are placed in a 500 □m fusion chamber and covered with 4 ml of 26° C.-27° C. fusion medium (0.3M mannitol, 0.1 mM MgSO₄, 0.05 mM CaCl₂). The cells are then electrofused by application of a double direct current (DC) electrical pulse of 70-100V for about 15 □s, approximately is apart. After fusion, the resultant fused reconstituted cells are then placed in a suitable medium until activation, eg., TCM-199 medium.

In a preferred method of cell fusion the donor animal cell is firstly attached to the enucleated oocyte. For example, a compound is selected to attach the animal cell to the enucleated oocyte to enable fusing of the animal cell and enucleated oocyte membranes. The compound may be any compound capable of agglutinating cells. The compound may be a protein or glycoprotein capable of binding or agglutinating carbohydrate. More preferably the compound is a lectin. The lectin may be selected from the group including Concanavalin A, Canavalin A, Ricin, soybean lectin, lotus seed lectin and phytohemaglutinin (PHA). Preferably the compound is PHA.

In one preferred embodiment, the method of electrofusion described above also comprises a further fusion step, or the fusion step comprises described above comprises one donor animal cell and two or more enucleated oocytes. The double fusion method has the advantageous effect of increasing the cytoplasmic volume of the reconstituted cell.

A reconstituted animal cell is typically activated by electrical and/or non-electrical means before, during, and/or after fusion of the nuclear donor and recipient oocyte (See, for example, Susko-Parrish et al., U.S. Pat. No. 5,496,720). Activation methods include:

1). Electric pulses;

2). Chemically induced shock;

3). Penetration by sperm;

4). Increasing levels of divalent cations in the oocyte by introducing divalent cations into the oocyte cytoplasm, eg., magnesium, strontium, barium or calcium, eg., in the form of an ionophore. Other methods of increasing divalent cation levels include the use of electric shock, treatment with ethanol and treatment with caged chelators; and

5). Reducing phosphorylation of cellular proteins in the oocyte by known methods, eg., by the addition of kinase inhibitors, eg., serine-threonine kinase inhibitors, such as 6-dimethyl-aminopurine, staurosporine, 2-aminopurine, and sphingosine. Alternatively, phosphorylation of cellular proteins may be inhibited by introduction of a phosphatase into the oocyte, eg., phosphatase 2A and phosphatase 2B.

The activated reconstituted animal cells, or embryos, are typically cultured in medium well known to those of ordinary skill in the art, and include, without limitation, TCM-199 plus 10% FSC, Tyrodes-Albumin-Lactate-Pyruvate (TALP), Ham's F-10 plus 10% FCS, synthetic oviductal fluid (“SOF”), B2, CRlaa, medium and high potassium simplex medium (“KSOM”).

The reconstituted cell may also be activated by known methods. Such methods include, eg., culturing the reconstituted cell at sub-physiological temperature, in essence by applying a cold, or actually cool temperature shock to the reconstituted cell. This may be most conveniently done by culturing the reconstituted animal cell at room temperature, which is cold relative to the physiological temperature conditions to which embryos are normally exposed. Suitable oocyte activation methods are the subject of U.S. Pat. No. 5,496,720, to Susko-Parrish et al., herein incorporated by reference in its entirety.

The activated reconstituted animal cells may then be cultured in a suitable in vitro culture medium until the generation of cells and cell colonies. Culture media suitable for culturing and maturation of animal embryos are well known in the art. Examples of known media, which may be used for bovine embryo culture and maintenance, include Ham's F-10 plus 10% FCS, TCM-199 plus 10% FCS, Tyrodes-Albumin-Lactate-Pyruvate (TALP), Dulbecco's Phosphate Buffered Saline (PBS), Eagle's and Whitten's media. One of the most common media used for the collection and maturation of oocytes is TCM-199, and 1 to 20% serum supplement including fetal calf serum, newborn serum, estrual cow serum, lamb serum or steer serum. A preferred maintenance medium includes TCM-199 with Earl salts, 10% FSC, 0.2 mM Na pyruvate and 50 □g/ml gentamicin sulphate. Any of the above may also involve co-culture with a variety of cell types such as granulosa cells, oviduct cells, BRL cells and uterine cells and STO cells.

Afterward, the cultured reconstituted animal cell or embryos are preferably washed and then placed in a suitable media, eg., TCM-199 medium containing 10% FCS contained in well plates which preferably contain a suitable confluent feeder layer. Suitable feeder layers include, by way of example, fibroblasts and epithelial cells, eg., fibroblasts and uterine epithelial cells derived from ungulates, chicken fibroblasts, murine (eg., mouse or rat) fibroblasts, STO and SI-m220 feeder cell lines, and BRL cells.

In one embodiment, the feeder cells comprise mouse embryonic fibroblasts. Preparation of a suitable fibroblast feeder layers are well known in the art.

The reconstituted animal cells are cultured on the feeder layer until the reconstituted cells reach a size suitable for transferring to a recipient female, or for obtaining cells which may be used to produce cells or cell colonies. Preferably, these reconstituted cells will be cultured until at least about 2 to 400 cells, more preferably about 4 to 128 cells, and most preferably at least about 50 cells. The culturing will be effected under suitable conditions, i.e., about 39° C. and 5% CO₂, with the culture medium changed in order to optimise growth typically about every 2-5 days, preferably about every 3 days.

The methods for embryo transfer and recipient animal management in the present invention are standard procedures used in the embryo transfer industry. Synchronous transfers are important for success of the present invention, ie., the stage of the nuclear transfer embryo is in synchrony with the estrus cycle of the recipient female. This advantage and how to maintain recipients are reviewed in Siedel, G. E., Jr. (“Critical review of embryo transfer procedures with cattle” in Fertilization and Embryonic Development in Vitro (1981) L. Mastroianni, Jr. and J. D. Biggers, ed., Plenum Press, New York, N.Y., page 323), the contents of which are hereby incorporated by reference.

Briefly, blastocysts may be transferred non-surgically or surgically into the uterus of a synchronised recipient. Other medium may also be employed using techniques and media well-known to those of ordinary skill in the art. In one procedure, cloned embryos are washed three times with fresh KSOM and cultured in KSOM with 0.1% BSA for 4 days and subsequently with 1% BSA for an additional 3 days, under 5% CO₂, 5% O₂ and 90% N₂ at 39° C. Embryo development is examined and graded by standard procedures known in the art. Cleavage rates are recorded on day 2 and cleaved embryos are cultured further for 7 days. On day seven, blastocyst development is recorded and one or two embryos, pending availability of embryos and/or animals, is transferred non-surgically into the uterus of each synchronised foster mother.

Foster mothers preferably are examined for pregnancy by rectal palpation or ultrasonography periodically, such as on days 40, 60, 90 and 120 of gestation. Careful observations and continuous ultrasound monitoring (monthly) preferably is made throughout pregnancy to evaluate embryonic loss at various stages of gestation. Any aborted fetuses should be harvested, if possible, for DNA typing to confirm clone status as well as routine pathological examinations.

The reconstituted animal cell, activated reconstituted animal cell, fetus and animal produced during the steps of such method, and cells, nuclei, and other cellular components which may be harvested therefrom, are also asserted as embodiments of the present invention. It is particularly preferred that the term animal produced be a viable animal.

The present invention can also be used to produce embryos, fetuses or offspring which can be used, for example, in cell, tissue and organ transplantation. By taking a fetal or adult cell from an animal and using it in the cloning procedure a variety of cells, tissues and possibly organs can be obtained from cloned fetuses as they develop through organogenesis. Cells, tissues, and organs can be isolated from cloned offspring as well. This process can provide a source of “materials” for many medical and veterinary therapies including cell and gene therapy. If the cells are transferred back into the animal in which the cells were derived, then immunological rejection is averted. Also, because many cell types can be isolated from these clones, other methodologies such as hematopoietic chimerism can be used to avoid immunological rejection among animals of the same species as well as between species.

In one embodiment, the candidate gene will be a plant gene which is capable of producing hybrid vigour as discussed supra.

Traditionally a hybrid plant is produced by random event by crossing one elite inbred plant with one or more other, genetically different and diverse, inbred plants. The crossing consists of taking the pollen from one inbred elite plant and transferring to the other elite inbred plant. The seed from crossing of two inbreds is a first generation hybrid and is called a F₁. The F₁ of commercially valuable inbreds have better yields, standability, and improvement in other important characteristics than either of the parents.

In the present invention once a candidate gene has been identified it is isolated or synthesised and either subcloned into an expression vector or transformed directly into a recipient plant.

There are many methods for transforming DNA segments into cells, but not all are suitable for delivering DNA to plant cells. Suitable methods for use with the current invention are believed to include virtually any method by which DNA can be introduced into a cell, such as by direct delivery of DNA such as by PEG-mediated transformation of protoplasts (Omirulleh et al., 1993), by desiccation/inhibition-mediated DNA uptake (Potrykus et al., 1985), by electroporation (U.S. Pat. No. 5,384,253, specifically incorporated herein by reference in its entirety), by agitation with silicon carbide fibers (Kaeppler et al., 1990; U.S. Pat. No. 5,302,523, specifically incorporated herein by reference in its entirety; and U.S. Pat. No. 5,464,765, specifically incorporated herein by reference in its entirety), by Agrobacterium-mediated transformation (U.S. Pat. No. 5,591,616 and U.S. Pat. No. 5,563,055; both specifically incorporated herein by reference) and by acceleration of DNA coated particles (U.S. Pat. No. 5,550,318; U.S. Pat. No. 5,538,877; and U.S. Pat. No. 5,538,880; each specifically incorporated herein by reference in its entirety), etc. Through the application of techniques such as these, plant cells may be stably transformed, and these cells developed into transgenic plants. In certain embodiments, acceleration methods are preferred and include, for example, microprojectile bombardment and the like.

Where one wishes to introduce candidate gene DNA by means of electroporation, it is contemplated that the method of Krzyzek et al. (U.S. Pat. No. 5,384,253, incorporated herein by reference in its entirety) will be particularly advantageous. In this method, certain cell wall-degrading enzymes, such as pectin-degrading enzymes, are employed to render the target recipient cells more susceptible to transformation by electroporation than untreated cells. Alternatively, recipient cells are made more susceptible to transformation by mechanical wounding.

To effect transformation by electroporation, one may employ either friable tissues, such as a suspension culture of cells or embryogenic callus or alternatively one may transform immature embryos or other organised tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; D'Halluin et al., 1992; Rhodes et al., 1995), wheat (Zhou et al., 1993), tomato (Hou and Lin, 1996), soybean (Christou et al., 1987), and tobacco (Lee et al., 1989).

One may also employ protoplasts for electroporation transformation of plants (Bates, 1994; Lazzeri, 1995). For example, the generation of transgenic soybean plants by electroporation of cotyledon-derived protoplasts is described by Dhir and Widholm in Intl. Patent Appl. Publ. No. WO 9217598 (specifically incorporated herein by reference). Other examples of species for which protoplast transformation has been described include barley (Lazerri, 1995), sorghum (Battraw et al., 1991), maize (Bhattacharjee et al., 1997), wheat (He et al., 1994), tomato (Tsukada, 1989), and soybean (Dhir et al., 1992).

A preferred method for delivering transforming candidate gene DNA segments to plant cells in accordance with the invention is microprojectile bombardment (U.S. Pat. No. 5,550,318; U.S. Pat. No. 5,538,880; U.S. Pat. No. 5,610,042; and PCT Application WO 94/09699; each of which is specifically incorporated herein by reference in its entirety). In this method, particles may be coated with nucleic acids and delivered into cells by a propelling force. Exemplary particles include those comprised Of tungsten, platinum, and preferably, gold. It is contemplated that in some instances candidate gene DNA precipitation onto metal particles would not be necessary for DNA delivery to a recipient cell using microprojectile bombardment. However, it is contemplated that particles may contain DNA rather than be coated with DNA. Hence, it is proposed that DNA-coated particles may increase the level of DNA delivery via particle bombardment but are not, in and of themselves, necessary.

An illustrative embodiment of a method for delivering DNA into plant cells by acceleration is the Biolistics Particle Delivery System, which can be used to propel particles coated with DNA or cells through a screen, such as a stainless steel or Nytex screen, onto a filter surface covered with plant cells cultured in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

Microprojectile bombardment techniques are widely applicable, and may be used to transform virtually any plant species. Examples of species for which have been transformed by microprojectile bombardment include monocot species such as maize (PCT Application WO 95/06128), barley (Ritala et al., 1994; Hensgens et al., 1993), wheat (U.S. Pat. No. 5,563,055, specifically incorporated herein by reference in its entirety), rice (Hensgens et al., 1993), oat (Torbet et al., 1995; Torbet et al., 1998), rye (Hensgens et al., 1993), sugarcane (Bower et al., 1992), and sorghum (Casa et al., 1993; Hagio et al., 1991); as well as a number of dicots including tobacco (Tomes et al., 1990; Buising and Benbow, 1994), soybean (U.S. Pat. No. 5,322,783, specifically incorporated herein by reference in its entirety), sunflower (Knittel et al. 1994), peanut (Singsit et al., 1997), cotton (McCabe and Martinell, 1993), tomato (VanEck et al. 1995), and legumes in general (U.S. Pat. No. 5,563,055, specifically incorporated herein by reference in its entirety).

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate. If desired, one or more screens may be positioned between the acceleration device and the cells to be bombarded.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described by Fraley et al., (1985), Rogers et al., (1987) and U.S. Pat. No. 5,563,055, specifically incorporated herein by reference in its entirety.

Agrobacterium-mediated transformation is most efficient in dicotyledonous plants and is the preferable method for transformation of dicots, including Arabidopsis, tobacco, tomato, and potato. Indeed, while Agrobacterium-mediated transformation has been routinely used with dicotyledonous plants for a number of years, it has only recently become applicable to monocotyledonous plants. Advances in Agrobacterium-mediated transformation techniques have now made the technique applicable to nearly all monocotyledonous plants. For example, Agrobacterium-mediated transformation techniques have now been applied to rice (Hiei et al., 1997; Zhang et al., 1997; U.S. Pat. No. 5,591,616, specifically incorporated herein by reference in its entirety), wheat (McCormac et al., 1998), barley (Tingay et al., 1997; McCormac et al., 1998), and maize (Ishidia et al., 1996).

Modern Agrobacterium transformation vectors are capable of replication in E. coli as well as Agrobacterium, allowing for convenient manipulations as described (Klee et al., 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described (Rogers et al., 1987) have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant strains where Agrobacterium-mediated transformation is efficient, it is the method of choice because of the facile and defined nature of the gene transfer.

Transformation of plant protoplasts can be achieved using methods based on calcium phosphate precipitation, polyethylene glycol treatment, electroporation, and combinations of these treatments (see, e.g., Potrykus et al., 1985; Lorz et al., 1985; Omirulleh et al., 1993; Fromm et al., 1986; Uchimiya et al., 1986; Callis et al., 1987; Marcotte et al., 1988).

Application of these systems to different plant strains depends upon the ability to regenerate that particular plant strain from protoplasts. Illustrative methods for the regeneration of cereals from protoplasts have been described (Fujimara et al., 1985; Toriyama et al., 1986; Yamada et al., 1986; Abdullah et al., 1986; Omirulleh et al., 1993 and U.S. Pat. No. 5,508,184; each specifically incorporated herein by reference in its entirety).

Examples of the use of direct uptake transformation of cereal protoplasts include transformation of rice (Ghosh-Biswas et al., 1994), sorghum (Battraw and Hall, 1991), barley (Lazerri, 1995), oat (Zheng and Edwards, 1990) and maize (Omirulleh et al., 1993).

To transform plant strains that cannot be successfully regenerated from protoplasts, other ways to introduce DNA into intact cells or tissues can be utilised. For example, regeneration of cereals from immature embryos or explants can be effected as described (Vasil, 1989). Also, silicon carbide fiber-mediated transformation may be used with or without protoplasting (Kaeppler, 1990; Kaeppler et al., 1992; U.S. Pat. No. 5,563,055, specifically incorporated herein by reference in its entirety). Transformation with this technique is accomplished by agitating silicon carbide fibers together with cells in a DNA solution. DNA passively enters as the cells are punctured. This technique has been used successfully with, for example, the monocot cereals maize (PCT Application WO 95/06128, specifically incorporated herein by reference in its entirety; Thompson, 1995) and rice (Nagatani, 1997).

Optimisation of Microprojectile Bombardment

For microprojectile bombardment transformation in accordance with the current invention, both physical and biological parameters may be optimised. Physical factors are those that involve manipulating the DNA/microprojectile precipitate or those that affect the flight and velocity of either the macro- or microprojectiles. Biological factors include all steps involved in manipulation of cells before and immediately after bombardment, such as the osmotic adjustment of target cells to help alleviate the trauma associated with bombardment, the orientation of an immature embryo or other target tissue relative to the particle trajectory, and also the nature of the transforming DNA, such as linearized DNA or intact supercoiled plasmids. It is believed that pre-bombardment manipulations are especially important for successful transformation of immature embryos.

Accordingly, it is contemplated that one may wish to adjust various of the bombardment parameters in small scale studies to fully optimise the conditions. One may particularly wish to adjust physical parameters such as DNA concentration, gap distance, flight distance, tissue distance, and helium pressure. It is further contemplated that the grade of helium may effect transformation efficiency. For example, differences in transformation efficiencies may be witnessed between bombardments using industrial grade (99.99% pure) or ultra pure helium (99.999% pure), although it is not currently clear which is more advantageous for use in bombardment. One may also optimize the trauma reduction factors (TRFs) by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation.

Both physical and biological parameters for bombardment may be addressed for further optimisation of ballistic transformation. Physical factors are those that involve manipulating the DNA/microprojectile precipitate or those that affect the flight and velocity of either the macro-or microprojectiles. Biological factors include all steps involved in manipulation of cells immediately before and after bombardment. The pre-bombardment culturing conditions, such as osmotic environment, the bombardment parameters, and the plasmid configuration have been adjusted to yield the maximum numbers of stable transformants.

(i) Physical Parameters

1. Gap Distance

The variable nest (macro holder) can be adjusted to vary the distance between the rupture disk and the macroprojectile, i.e., the gap distance. This distance can be varied from 0 to 2 cm. The predicted effects of a shorter gap are an increase of velocity of both the macro-and microprojectiles, an increased shock wave (which leads to tissue splattering and increased tissue trauma), and deeper penetration of microprojectiles. Longer gap distances would have the opposite effects but may increase viability and therefore the total number of recovered stable transformants.

2. Flight Distance

The fixed nest (contained within the variable nest) can be varied between 0.5 and 2.25 cm in predetermined 0.5 cm increments by the placement of spacer rings to adjust the flight path traversed by the macroprojectile. Short flight paths allow for greater stability of the macroprojectile in flight but reduce the overall velocity of the microprojectiles. Increased stability in flight increases, for example, the number of centered GUS foci. Greater flight distances (up to some point) increase velocity but also increase instability in flight. Based on observations, it is recommended that bombardments typically be done with a flight path length of about 1.0 cm to 1.5 cm.

3. Tissue Distance

Placement of tissue within the gun chamber can have significant effects on microprojectile penetration. Increasing the flight path of the microprojectiles will decrease velocity and trauma associated with the shock wave. A decrease in velocity also will result in shallower penetration of the microprojectiles.

4. Helium Pressure

By manipulation of the type and number of rupture disks, pressure can be varied between 400 and 2000 psi within the gas acceleration tube. Optimum pressure for stable transformation has been determined to be between 1000 and 1200 psi.

5. Coating of Microprojectiles.

For microprojectile bombardment, one will attach (i.e. “coat”) DNA to the microprojectiles such that it is delivered to recipient cells in a form suitable for transformation thereof. In this respect, at least some of the transforming DNA must be available to the target cell for transformation to occur, while at the same time during delivery the DNA must be attached to the microprojectile. Therefore, availability of the transforming DNA from the microprojectile may comprise the physical reversal of bonds between transforming DNA and the microprojectile following delivery of the microprojectile to the target cell. This need not be the case, however, as availability to a target cell may occur as a result of breakage of unbound segments of DNA or of other molecules which comprise the physical attachment to the microprojectile. Availability may further occur as a result of breakage of bonds between the transforming DNA and other molecules, which are either directly or indirectly attached to the microprojectile. It further is contemplated that transformation of a target cell may occur by way of direct recombination between the transforming DNA and the genomic DNA of the recipient cell. Therefore, as used herein, a “coated” microprojectile will be one which is capable of being used to transform a target cell, in that the transforming DNA will be delivered to the target cell, yet will be accessible to the target cell such that transformation may occur.

Any technique for coating microprojectiles which allows for delivery of transforming DNA to the target cells may be used. Methods for coating microprojectiles which have been demonstrated to work well with the current invention have been specifically disclosed herein. DNA may be bound to microprojectile particles using alternative techniques, however. For example, particles may be coated with streptavidin and DNA end labelled with long chain thiol cleavable biotinylated nucleotide chains. The DNA adheres to the particles due to the streptavidin-biotin interaction, but is released in the cell by reduction of the thiol linkage through reducing agents present in the cell.

Alternatively, particles may be prepared by functionalising the surface of a gold oxide particle, providing free amine groups. DNA, having a strong negative charge, binds to the functionalised particles.

Furthermore, charged particles may be deposited in controlled arrays on the surface of mylar flyer disks used in the PDS-1000 Biolistics device, thereby facilitating controlled distribution of particles delivered to target tissue.

As disclosed above, it further is proposed, that the concentration of DNA used to coat microprojectiles may influence the recovery of transformants containing a single copy of the transgene. For example, a lower concentration of DNA may not necessarily change the efficiency of the transformation, but may instead increase the proportion of single copy insertion events. In this regard, approximately 1 ng to 2000 ng of transforming DNA may be used per each 1.8 mg of starting microprojectiles. In other embodiments of the invention, approximately 2.5 ng to 1000 ng, 2.5 ng to 750 ng, 2.5 ng to 500 ng, 2.5 ng to 250 ng, 2.5 ng to 100 or 2.5 ng to 50 ng of transforming DNA may be used per each 1.8 mg of starting microprojectiles.

Various other methods may also be used to increase transformation efficiency and/or increase the relative proportion of low-copy transformation events. For example, the inventors contemplate end-modifying transforming DNA with alkaline phosphatase or an agent which will blunt DNA ends prior to transformation. Still further, an inert carrier DNA may be included with the transforming DNA, thereby lowering the effective transforming DNA concentration without lowering the overall amount of DNA used. These techniques are further described in U.S. patent application Ser. No. 08/995,451, filed Dec. 22, 1997, the disclosure of which is specifically incorporated herein by reference in its entirety.

(ii) Biological Parameters

Culturing conditions and other factors can influence the physiological state of the target cells and may have profound effects on transformation and integration efficiencies. First, the act of bombardment could stimulate the production of ethylene which could lead to senescence of the tissue. The addition of antiethylene compounds could increase transformation efficiencies. Second, it is proposed that certain points in the cell cycle may be more appropriate for integration of introduced DNA. Hence synchronisation of cell cultures may enhance the frequency of production of transformants. For example, synchronisation may be achieved using cold treatment, amino acid starvation, or other cell cycle-arresting agents. Third, the degree of tissue hydration also may contribute to the amount of trauma associated with bombardment as well as the ability of the microprojectiles to penetrate cell walls.

The position and orientation of an embryo or other target tissue relative to the particle trajectory may also be important. For example, the PDS-1000 biolistics device does not produce a uniform spread of particles over the surface of a target petri dish. The velocity of particles in the centre of the plate is higher than the particle velocity at further distances from the centre of the petri dish. Therefore, it is advantageous to situate target tissue on the petri dish such as to avoid the centre of the dish, referred to by some as the “zone of death.” Furthermore, orientation of the target tissue with regard to the trajectory of targets also can be important. It is contemplated that it is desirable to orient the tissue most likely to regenerate a plant toward the particle stream. For example, the scutellum of an immature embryo comprises the cells of greatest embryogenic potential and therefore should be oriented toward the particle stream.

It also has been reported that slightly plasmolyzed yeast cells allow increased transformation efficiencies (Armaleo et al., 1990). It was hypothesised that the altered osmotic state of the cells helped to reduce trauma associated with the penetration of the microprojectile. Additionally, the growth and cell cycle stage may be important with respect to transformation.

1. Osmotic Adjustment

It has been suggested that osmotic pre-treatment could potentially reduce bombardment associated injury as a result of the decreased turgor pressure of the plasmolyzed cell. In a previous study, the number of cells transiently expressing GUS increased following subculture into both fresh medium and osmotically adjusted medium (PCT Application WO 95/06128, specifically incorporated herein by reference in its entirety). Pretreatment times of 90 minutes showed higher numbers of GUS expressing foci than shorter times. Cells incubated in 500 mOSM/kg medium for 90 minutes showed an approximately 3.5 fold increase in transient GUS foci than the control. Preferably, immature embryos are precultured for 4-5 hours prior to bombardment on culture medium containing 12% sucrose. A second culture on 12% sucrose is performed for 16-24 hours following bombardment. Alternatively, type II cells are pretreated on 0.2M mannitol for 3-4 hours prior to bombardment. It is contemplated that pretreatment of cells with other osmotically active solutes for a period of 1-6 hours may also be desirable.

2. Plasmid Configuration

In some instances, it will be desirable to deliver candidate gene DNA to cells that do not contain DNA sequences necessary for maintenance of the plasmid vector in the bacterial host, eg., E. coli, such as antibiotic resistance genes, including but not limited to ampicillin, kanamycin, and tetracycline resistance, and prokaryotic origins of DNA replication. In such case, a DNA fragment containing the transforming DNA may be purified prior to transformation. An exemplary method of purification is gel electrophoresis on a 1.2% low melting temperature agarose gel, followed by recovery from the agarose gel by melting gel slices in a 6-10 fold excess of Tris-EDTA buffer (10 mM Tris-HCl pH 8.0, 1 mM EDTA, 70° C.-72° C.); frozen and thawed (37° C.); and the agarose pelleted by centrifugation. A Qiagen Q-100 column then may be used for purification of DNA. For efficient recovery of DNA, the flow rate of the column may be adjusted to 40 ml/hr.

Isolated DNA fragments can be recovered from agarose gels using a variety of electroelution techniques, enzyme digestion of the agarose, or binding of DNA to glass beads (e.g., Gene Clean). In addition, HPLC and/or use of magnetic particles may be used to isolate DNA fragments. As an alternative to isolation of DNA fragments, a plasmid vector can be digested with a restriction enzyme and this DNA delivered to maize cells without prior purification of the expression cassette fragment.

Tissue culture requires media and controlled environments. “Media” refers to the numerous nutrient mixtures that are used to grow cells in vitro, that is, outside of the intact living organism. The medium usually is a suspension of various categories of ingredients (salts, amino acids, growth regulators, sugars, buffers) that are required for growth of most cell types. However, each specific cell type requires a specific range of ingredient proportions for growth, and an even more specific range of formulas for optimum growth. Rate of cell growth also will vary among cultures initiated with the array of media that permit growth of that cell type.

Nutrient media is prepared as a liquid, but this may be solidified by adding the liquid to materials capable of providing a solid support. Agar is most commonly used for this purpose. Bactoagar, Hazelton agar, Gelrite, and Gelgro are specific types of solid support that are suitable for growth of plant cells in tissue culture.

Some cell types will grow and divide either in liquid suspension or on solid media. As disclosed herein, maize cells will grow in suspension or on solid medium, but regeneration of plants from suspension cultures requires transfer from liquid to solid media at some point in development. The type and extent of differentiation of cells in culture will be affected not only by the type of media used and by the environment, for example, pH, but also by whether media is solid or liquid.

Recipient cell targets include, but are not limited to, meristem cells, including the shoot apex (U.S. Pat. No. 5,736,369), Type I, Type II, and Type III callus, immature embryos and gametic cells such as microspores, pollen, sperm and egg cells. It is contemplated that any cell from which a fertile plant may be regenerated is useful as a recipient cell. Type I, Type II, and Type III callus may be initiated from tissue sources including, but not limited to, immature embryos, seedling apical meristems, microspores and the like. Those cells which are capable of proliferating as callus are also recipient cells for genetic transformation. The present invention provides techniques for transforming immature embryos and subsequent regeneration of fertile transgenic plants. Transformation of immature embryos obviates the need for long term development of recipient cell cultures. Pollen, as well as its precursor cells, microspores, may be capable of functioning as recipient cells for genetic transformation, or as vectors to carry foreign DNA for incorporation during fertilisation. Direct pollen transformation would obviate the need for cell culture. Meristematic cells (i.e., plant cells capable of continual cell division and characterised by an undifferentiated cytological appearance, normally found at growing points or tissues in plants such as root tips, stem apices, lateral buds, etc.) may represent another type of recipient plant cell. Because of their undifferentiated growth and capacity for organ differentiation and totipotency, a single transformed meristematic cell could be recovered as a whole transformed plant. In fact, it is proposed that embryogenic suspension cultures may be an in vitro meristematic cell system, retaining an ability for continued cell division in an undifferentiated state, controlled by the media environment.

Cultured plant cells that can serve as recipient cells for transforming with desired DNA segments may be any plant cells including corn cells, and more specifically, cells from Zea mays L. Somatic cells are of various types. Embryogenic cells are one example of somatic cells which may be induced to regenerate a plant through embryo formation. Non-embryogenic cells are those which typically will not respond in such a fashion. An example of non-embryogenic cells are certain Black Mexican Sweet (BMS) corn cells.

The development of embryogenic calli and suspension cultures useful in the context of the present invention, eg., as recipient cells for transformation, has been described in U.S. Pat. No. 5,134,074; and U.S. Pat. No. 5,489,520; each of which is incorporated herein by reference in its entirety.

Certain techniques may be used that enrich recipient cells within a cell population. For example, Type II callus development, followed by manual selection and culture of friable, embryogenic tissue, generally results in an enrichment of recipient cells for use in, microprojectile transformation. Suspension culturing, particularly using the media disclosed herein, may improve the ratio of recipient to non-recipient cells in any given population. Manual selection techniques which can be employed to select recipient cells may include, eg., assessing cell morphology and differentiation, or may use various physical or biological means of selection. Cryopreservation is also a possible method of selecting for recipient cells.

Manual selection of recipient cells, eg., by selecting embryogenic cells from the surface of a Type II callus, is one means that may be used in an attempt to enrich for recipient cells prior to culturing (whether cultured on solid media or in suspension). The preferred cells may be those located at the surface of a cell cluster, and may further be identifiable by their lack of differentiation, their size and dense cytoplasm. The preferred cells will generally be those cells which are less differentiated, or not yet committed to differentiation. Thus, one may wish to identify and select those cells which are cytoplasmically dense, relatively unvacuolated with a high nucleus to cytoplasm ratio (e.g., determined by cytological observations), small in size (e.g., 10-20 □m), and capable of sustained divisions and somatic proembryo formation.

It is proposed that other means for identifying such cells may also be employed. For example, through the use of dyes, such as Evan's blue, which are excluded by cells with relatively non-permeable membranes, such as embryogenic cells, and taken up by relatively differentiated cells such as root-like cells and snake cells (so-called due to their snake-like appearance).

Other possible means of identifying recipient cells include the use of isozyme markers of embryogenic cells, such as glutamate dehydrogenase, which can be detected by cytochemical stains (Fransz et al., 1989). However, it is cautioned that the use of isozyme markers including glutamate dehydrogenase may lead to some degree of false positives from non-embryogenic cells such as rooty cells which nonetheless have a relatively high metabolic activity.

In one embodiment, the candidate gene, rather than producing hybrid vigour, will overcome, treat or at least alleviate the symptoms of disease. It will be appreciated by those skilled in the field that the experimental protocols outline above may be used to bring about this outcome.

Generally, the terms “treating,” “treatment” and the like are used herein to mean affecting an individual or subject, their tissue or cells to obtain a desired pharmacological and/or physiological effect. The effect may be prophylactic in terms of completely or partially preventing the a disease or sign or symptom thereof, and/or may be therapeutic in terms of a partial or complete cure of disease. “Treating” as used herein covers any treatment of, or prevention of disease in a plant or animal, and includes: (a) preventing the disease from occurring in a plant or animal that may be predisposed to the disease, but has not yet been diagnosed as having them; (b) inhibiting the disease, ie., arresting its development; or (c) relieving or ameliorating the symptoms of the disease, ie., cause regression of the symptoms of the disease.

Once a plant or animal afflicted with a disease has been diagnosed and a candidate gene, or combination of genes, has been identified then these genes or gene products may be administered to the plant or animal either using the techniques described above with respect to hybrid vigour or using standard medical or agricultural techniques.

The terms “administration,” administering,” and “administered” are used herein interchangeably. For example, the candidate gene products may be administered orally including sublingual, topically, or parenterally in dosage unit formulations containing conventional non-toxic pharmaceutically acceptable carriers, adjuvants, and vehicles. The term parenteral as used herein includes subcutaneous injections, aerosol, intravenous, intramuscular, intrathecal, intracranial, injection or infusion techniques or rectal or vaginally.

Throughout the specification, unless the context requires otherwise, the word “comprise” or variations such as “comprises” or “comprising”, will be understood to imply the inclusion of a stated integer or group of integers but not the exclusion of any other integer or group of integers.

### IDENTIFICATION OF A NOVEL BIOCHEMICAL PATHWAY THAT FORMS HYBRID mRNA MOLECULES IN HETEROZYGOUS OFFSPRING

Two allelic forms of the gene DNMT2 have been described (Franchina et al., Hum. Hered. 52,210 (2001)). DNMT2 encodes an enzyme, which is involved in the process of DNA methylation. One of the alleles DNMT2I includes the nucleotides G in position 104 of exon 2 and C in position 50 of exon 4. The alternative allele, DNMT2II includes nucleotides A and T in these positions respectively. Thus the difference in the sequence of DNA within exons 2 and 4 between each of the two allelic forms of DNMT2 permits the allelic origin of exons 2 and 4 in a final mRNA molecule to be determined. These findings were used to test whether hybrid forms of mRNA can be formed in heterozygous offspring by incorporation of exons from each allele into the same mature mRNA molecule, thereby generating novel polypeptide or protein molecules unique to heterozygous offspring.

In order to determine whether and how efficiently exons or parts thereof from each DNMT2 allele can be incorporated into the same mature RNA molecule after splicing of the primary transcript, mRNA was isolated from the peripheral blood leukocytes (PBL) of two subjects III 1 and III 2, who were confirmed by segregation studies to be heterozygous for the two allelic forms of DNMT2 (see Franchina et al,. Hum. Hered. 52,210 (2001)).

The mRNA was subjected to RT-PCR using primers, which amplified a region of cDNA of approximately 480 bp that included exons 2 and 4 from DNMT2. The RT-PCR products of approximately 480 bp in length from subjects III 1 and III 2 were cloned and a series of clones from each RT-PCR product were sequenced.

As shown in Table 1, inserts from 13 of 15 clones sequenced from subject III 1 reflected bi-allelic expression of DNMT2 and as expected, cis-assembly of exons 2 and 4 in the mature mRNA molecule. One of the clones however, included a splice product that comprised exon 2 from the DNMT2 I allele and exon 4 from the DNMT2 II allele. A further clone comprised exon 2 from the DNMT2 II allele as well as exon 4 from the DNMT2 I allele. To assess the reproducibility of these findings and to exclude the possibility that the two apparent hybrid mRNA molecules found in subject III 1 were consequential to some form of mitotic recombinational event, mRNA species were examined similarly from the PBL of subject III 2.

As shown in Table 1, 11 of 14 clones contained inserts, which reflected bi-allelic expression of DNMT2 and cis-exonic assembly. The structure of a further 3 clones, however, indicated that hybrid mRNA molecules had been assembled. Two of them included exon 2 from the DNMT2 II allele and exon 4 from the DNMT2 I allele. The sequence of a further clone revealed that a spliced mRNA molecule had been assembled by incorporation of exon 2 from the DNMT2 I allele and exon 4 from the DNMT2 II allele.

mRNA was then isolated from the PBL of a further two subjects, II 3 and II 4 (Franchina et al., Hum. Hered. 52,210 (2001)) who were confirmed to be homozygous for each of the two alternative allelic forms of DNMT2. An in vitro prepared mixture of equal quantities of mRNA from subjects II 3 and II 4 was treated in the same way as mRNA from subjects III 1 and III 2 used to exclude the possibility that the hybrid mRNA DNMT2 molecules found in the PBL of subjects III 1 and III 2 may have been generated in vitro due to template switching in the PCR stage of the RT-PCR due to the formation of truncated reverse transcripts. As shown in Table 1, no hybrid mRNA molecules were found by reference to the sequence of inserts from 13 clones. Taken together, these findings establish that almost 20% of all spliced DNMT2 RNA molecules comprise hybrid forms.

TABLE 1
Distribution of spliced DNMT2 mRNA molecules
in peripheral blood leukocytes according
to the nucleotides present at the
polymorphic positions within exons 2 and 4.

| DNMT2 transcripts | | | Subject | | |
|---|---|---|---|---|---|
| nt 104 of exon 2 | | nt 50 of exon 4 | III 1 | III 2 | Mix* |
| G | | C) | 13 | 11 | 13 |
| | or | ) | | | |
| A | | T) | | | |
| G | | T) | 2 | 3 | 0 |
| | or | ) | | | |

| | |
|---|---|
| A | C) |

Note:
The genotypes DNMT2 I/II were confirmed in subjects III 1, III 2, and DNMT2 I/I and DNMT2 II/II in subjects II 3 and II 4 respectively by pedigree analysis.
*A mixture of mRNA from the homozygous DNMT2 I and DNMT2 II parents of subjects III 1, III 2, (II 3 and II 4) was also subjected to RT-PCR and the products cloned and sequenced.
No inserts were obtained which specified G-T or A-C spliced DNMT2 mRNA molecules.
These results confirm that our findings reflect the presence of an in vivo primary transcript splicing system involved in trans-exonic bi-allelic assembly of spliced mRNA molecules.

### VALIDATION OF THE USE OF THE STRUCTURAL CRITERIA TO IDENTIFY GENES WHICH PROMOTE HV OR HD©

The invention will now be further described by way of reference only to the following non-limiting examples. It should be understood, however, that the examples following are illustrative only, and should not be taken in any way as a restriction on the generality of the invention described above. In particular, while the invention has been described in detail above, in relation to the identification of candidate genes using DNMT2 alleles, it will be clearly understood that the findings herein are not limited to this gene or alleles.

HV occurs when an offspring displays an enhanced form of any particular biological phenotype when compared to expression of the same biological phenotype in either of its parents. HD© occurs when an offspring displays a reduced form of any particular biological phenotype or disease when compared to its parents.

The biological phenotype of HD may be caused directly by a reduction in the activity of hybrid proteins such as enzymes. On the other hand, some hybrid proteins may be recognised as immunologically foreign and cause the onset of some forms of HD by inducing an auto-immune process. The invention disclosed herein explains how an enhanced or a reduced biological phenotype is generated in hybrid offspring by the formation of novel proteins. The hybrid offspring must inherit allelic forms of the relevant genes which must differ according to the structural criteria that have been defined supra.

### EXAMPLE 1

### Osteoporosis as a Model of HV in Humans

Osteoporosis is a bone disorder suffered by almost 40% of post-menopausal women. It is caused by increased bone resorption by osteoclasts resulting in decreased bone density and increased susceptibilit_{y} to bone fractures. Calcitonin is used to treat osteoporosis because it binds to the calcitonin receptor on osteoclasts and reduces their ability to cause bone decay.

Taboulet et al., Hum.Mol.Genet.7,2129(1998) have shown that subjects who are heterozygous for wild-type calcitonin receptor alleles, as shown in Table 2, have higher bone density and a reduced risk of bone fractures compared to subjects who are homozygous for the same wild type alleles.

Table 2 shows the exon location of nucleotide changes that encode different amino acids in different allelic forms of the calcitonin receptor. These coding sequence variations were retrieved from NCBI databases. As shown in the Table 2, exon 1 may encode either leucine or arginine at amino acid position 126 whereas proline or leucine may be encoded by nucleotide differences within exon 9 at amino acid position 447.The distribution of nucleotide sequence differences which encode the alternative amino acids at positions 126 and 447 of the calcitonin receptor satisfy the structural criteria required to enable the formation of novel or hybrid forms of calcitonin receptor in subjects who are heterozygous for different allelic forms of the calcitonin receptor gene.

According to the invention described herein, permitted combinations of amino acids at positions 126 and 447 within the calcitonin receptor comprise, (a) leucine/proline,(b) leucine/leucine,(c) arginine/proline and (d) arginine/leucine, two of which comprise wild-type alleles whereas the other two comprise hybrid forms of the calcitonin receptor which affect bone density and susceptibility to bone fracture.

TABLE 2
Exon location of nucleotide changes which encode
amino acid differences in alternative calcitonin
receptor alleles

| Amino acid position | Exon | Amino acid alternative |
|---|---|---|
| 126 | 1 | (L) Leucine |
| 126 | 1 | (R) Arginine |
| 447 | 9 | (P) Proline |
| 447 | 9 | (L) Leucine |

### EXAMPLE 2

### Argininosuccinic Aciduria as a Mammalian Model for New Biochemical Pathway

In humans, at least 5 different allelic forms of the gene which encodes the enzyme argininosuccinate lyase (ASL) have been identified. Some of them, ASL(D87G), ASL(Q286R) and ASL(A398D), for example, encode mutant forms of ASL. Subjects that are homozygous for each of these mutant forms of ASL produce an inactive form of the enzyme leading to the onset of a disease known as argininosuccinic aciduria.

It is well recognised that in offspring who inherit mutant allelic forms of ASL such as ASL(Q286R) and ASL(D87G)in heterozygous form from each of their affected parents, a form of HV is observed whereby activity of ASL is restored (see, for example, Walker et al., J. Biol. Chem. 272,6777 (1997) incorporated herein by reference). The alleles which encode ASL(D87G) and ASL(Q286R) are characterised by nucleotide differences which are located within different ASL exons. The nucleotide changes that generate aspartic acid or glycine at amino acid position 87 are located in exon 3 whereas the nucleotide changes that encode glutamine or arginine are located within exon 11. The localisation of nucleotide sequence differences which encode the alternative amino acids at positions 87 and 286 in exons 3 and 11 respectively satisfy the structural criteria required to permit the synthesis of normal ASL enzyme in subjects who are heterozygous for two different mutant forms of the ASL gene.

### EXAMPLE 3

### Cystinuria as a Model for New Biochemical Pathway

Cystinuria is a human disorder characterised by impaired renal absorption of cystine and other amino acids. One form of cystinuria (referred to as non-type 1 cystinuria) has been shown to be caused by mutations in a gene designated SLC7A9. Homozygosity for mutant forms of SLC7A9 such as G105R, V170M and A354T results in a marked reduction in cystine reabsorption. As shown by Font et al., Hum.Mol.Genet.10, 305(2001),heterozygosity for each of these mutant forms of SLC7A9 results in dramatic restoration of cystine reabsorption. In keeping with the model nucleotide changes within SLC7A9 which result in generation of the mutant alleles G105R, V170M and A354T, reside in exons 4, 5 and 10 respectively. The localisation of the nucleotide changes which encode each of the three mutant forms of SLC7A9 satisfy the structural requirements needed to synthesise normally active SLC7A9 in subjects who are heterozygous for any two of the three mutant forms of SLC7A9 shown above.

### EXAMPLE 4

### Diabetes as a Mammalian HD© Model For New Biochemical Pathway

Diabetes is a common serious disease in which blood glucose levels are unable to be controlled properly because of impaired insulin secretion or resistance to its action. There are many different mechanistic forms of diabetes. In type 1 diabetes (diabetes of the young) insulin release is impaired because of immune attack on the islet cells, the cells within the pancreas that secrete insulin. Glutamic acid decarboxylase (GAD 65) encodes a major islet cell auto-antigen targeted by the immune system in type 1 diabetes.

Table 3 details the exonic location of nucleotide changes that encode different amino acids in alternative allelic forms of the enzyme glutamic acid decarboxylase. Coding region sequence variations were retrieved from NCBI databases. As shown in Table 3, exon 1 includes nucleotide differences that encode arginine or glycine at amino acid position 12. Exon 4, on the other hand, includes nucleotide changes that encode either asparagine or lysine at amino acid position 124 as well as glutamine or proline at position 153. Table 3 also shows that the alternative amino acids glutamic acid or glycine at position 232 are encoded by nucleotide differences within exon 6, arginine or lysine at position 286 by nucleotide changes within exon 8, and either alanine or glycine at position 326 by nucleotide changes within exon 10.

The distribution of nucleotide sequence differences that encode alternative amino acids and that are included in exons 1, 4, 6, 8 or 10 satisfies the structural criteria that need to be fulfilled to generate hybrid forms of glutamic acid decarboxylase in subjects who are heterozygous for any two of the different allelic forms of the enzyme referred to in Table 3. For example, by reference to the alternative amino acids which can be encoded by nucleotide differences located in exons 6 and 8 only, the combinations of (a) glutamic acid/arginine (b) glutamic acid/lysine (c) glycine/arginine and (d) glycine/lysine at amino acid positions 232 and 286 are formed in accordance with the new biochemical pathway. Two of these combinations comprise wild-type enzymes whereas the other two comprise hybrid forms.

TABLE 3
Exon location of nucleotidechanges which encode
amino acid differences in alternative glutamic
acid decarboxylase (GAD65) alleles

| Amino acid position | Exon | Amino acid alternative |
|---|---|---|
| 12 | 1 | (R) Arginine |
| 12 | 1 | (G) Glycine |
| 124 | 4 | (N) Asparagine |
| 124 | 4 | (K) Lysine |
| 153 | 4 | (Q) Glutamine |
| 153 | 4 | (P) Proline |
| 232 | 6 | (E) Glutamic acid |
| 232 | 6 | (G) Glycine |
| 286 | 8 | (R) Arginine |
| 286 | 8 | (K) Lysine |
| 326 | 10 | (A) Alanine |
| 326 | 10 | (G) Glycine |

### The Role of GAD65 Heterozygosity in Autoantibody Induction

GAD65 auto-antibodies may be detected in the serum of patients suffering Type I diabetes prior to disease onset as well as in the serum of their asymptomatic sibs, albeit at lower levels.

In keeping with inventors' findings and hypothesis that heterozygosity for GAD65 alleles which fulfil the structural criteria allows generation of hybrid forms of GAD65 that enhance the immune response against GAD65, auto-antibody levels should vary according to GAD65 genotype even in non-type I diabetic subjects. Results of a recent comprehensive study by Boutin et al,. PLoS Biol. 1, 68 (2003) confirm that GAD65 auto-antibody levels are significantly higher in normal non-diabetic subjects as well as obese patients who are heterozygous for GAD65 alleles compared to the same groups of subjects and patients who are homozygous for the same GAD65 alleles. These findings strongly support inventors claim that novel polypeptides or proteins, formed by the novel biochemical pathway in subjects who are heterozygous for different forms of alleles, which fulfil inventors structural criteria, synthesise auto-antibodies more readily than subjects who are homozygous for the same GAD65 alleles.

On the other hand, types of diabetes which occur later in life, some of which are associated with obesity, are not caused by an auto-immune process. These forms of diabetes are referred to as type 2 diabetes. The most consistently recognised type 2 diabetes candidate gene located on chromosome 2 is calpain-10 (CALP-10). No mutant forms of CALP-10 have been found to explain the relationship between this gene and development of type 2 diabetes even though it is associated with the highest risk for development of type 2 diabetes. The HD model for the association of CALP-10 with susceptibility to development of type 2 diabetes is strongly supported by findings that many different allelic forms of CALP-10 have been identified, such as L34V, T504A, R555C,andV661I that satisfy the structural criteria that are required to form hybrid enzymes in heterozygous offspring via the novel biochemical pathway. In keeping with the model, increased susceptibility to development of type 2 diabetes has been shown to be associated with heterozygosity for different allelic forms of CALP-10(see within Cox et al., Diabetes 53,19(2004)).

Insulin-degradation enzyme (IDE) is another strong candidate type 2 diabetes susceptibility gene found to be located on chromosome 10 by genomic scanning. IDE is involved in the proteolytic degradation of insulin, most likely as part of termination of the insulin response. As with CALP-10, no disease causing mutational forms of IDE have been found.

The exonic location of nucleotide changes which encode different amino acids within the insulin degrading enzyme is shown in Table 4.The coding sequence variations were retrieved from NCBI databases. As shown in the Table, exon 3 contains nucleotide sequence differences which encode the alternative amino acids phenylalanine or leucine at position 298.Moreover, amino acids leucine or phenylalanine may be encoded at amino acid position 582 by nucleotide changes located within exon 5 and serine or glycine at amino acid position 854 may be encoded by nucleotide differences within exon 11.Asparagine or aspartic acid may be encoded at amino acid position 947 by variation in sequence included in exon 20.

The distribution of nucleotide sequence differences which encode alternative amino acids at positions 298, 582, 845, and 947 and which are located in exons 3, 5, 11 and 20 respectively satisfy the structural criteria required to synthesise hybrid forms of insulin degrading enzyme in subjects who are heterozygous for any two of the alternative alleles shown in Table 4.For example, with reference to alleles encoded by exons 11 and 20 only, amino acid combinations at positions 845 and 947 include (a) serine/asparagine (b) serine/aspartic acid (c) glycine/asparagine and (d) glycine/aspartic acid. Two of these combinations reflect wild-type enzymes whereas the other two comprise hybrid forms of the enzyme.

TABLE 4
Exon location of nucleotide changes which encode
amino acid differences in alternative insulin degrading
enzyme alleles.

| Amino acid position | Exon | Amino acid alternative |
|---|---|---|
| 298 | 3 | (F) Phenylalanine |
| 298 | 3 | (L) Leucine |
| 582 | 5 | (L) Leucine |
| 582 | 5 | (F) Phenylalanine |
| 845 | 11 | (S) Serine |
| 845 | 11 | (G) Glycine |
| 947 | 20 | (N) Asparagine |
| 947 | 20 | (D) Aspartic acid |

### EXAMPLE 5

### Plant Model Which Supports the Role of New Biochemical Pathway in HV

Catalase 1 (Cat-1) is an oxidoreductase type of enzyme that plays an important role in the breakdown of hydrogen peroxide in plants. In maize, at least 6 different allelic forms of Cat-1 have been identified, most of which differ in amino acid composition.

In 1972 a hybrid form of Cat-1 (Scandalios et al., Arch. Biochem. Biophys. 153, 695 (1972)) was identified in plants that were heterozygous for two different allelic forms of Cat-1. The hybrid Cat-1 enzyme was shown to possess a range of enhanced biochemical properties compared to either of the homozygous parental forms.

The amino acid changes defining alternative allelic forms of Cat-1 are instructed by nucleotide differences located in different exons within the Cat-1 gene. The new biochemical pathway provides a plausible explanation for the formation of the hybrid Cat-1 molecules with enhanced biochemical properties compared to wild-types that does not rely on the formation of polymeric forms of the enzyme.

### EXAMPLE 6

### Identification of Hybrid ASL Molecules

As outlined below, it is possible to prove that active argininosuccinate lyase(ASL) can be generated in offspring of affected parents by inclusion of wild-type encoding exons from each of the different mutant alleles into the same mRNA molecule by way of the newly identified biochemical pathway.

Previous studies have shown that when combinations of inactive ASL mutants such as ASL(D87G) and ASL(Q286R) are inherited in heterozygous form, activity is partially restored. Therefore, mRNA can be isolated from fibroblasts or liver of a subject who is proven to have inherited each of the two inactive allelic mutant forms of ASL. Total mRNA can then be subjected to RT-PCR using oligonucleotide primers that span individual exons. The RT-PCR products containing these regions are then cloned and sequenced.

Approximately 85% of clones generated from the RT-PCR products from the subject genotyped as ASL(D87G)/(Q286R) would confirm firstly, bi-allelic expression of ASL and cis-assembly of exons. The sequence of inserts from approximately 15% of clones however would include sequences from each of the two parental alleles in the same clone and thus generate approximately 15% of wild-type enzyme accounting for restoration of enzyme activity.

### EXAMPLE 7

### Association of Hybrid GAD65 Molecules with GAD65 Auto-Antibody Production

Total mRNA can be extracted from islet cells of pancreatic tissue obtained from a patient suffering type 1 diabetes. If the patient is heterozygous for GAD65 alleles, which have nucleotide changes in different exons which encode non-synonymous amino acids (see Table 3), the patient would have high titre GAD65 auto-antibody in his/her blood.

Total pancreatic mRNA can then be subjected to RT-PCR using oligonucleotide primers which span exons 1, 4, 6, 8 or 10, according to the patient's GAD65 genotype.

It would be expected that the sequence of inserts of approximately 85% of clones would confirm bi-allelic expression and cis-assembly of exons from each of the two alleles. The sequence of a further 15% of clones would be expected to contain inserts that confirm that a hybrid form of GAD65 is synthesised in islet cells from a patient suffering type 1 diabetes who is heterozygous for different allelic forms of GAD65 as defined supra and has circulating GAD65 auto-antibodies in their blood.

The same experimental rationale is applicable to all species when it becomes known that the offspring under investigation has inherited genes that have alleles as defined supra and the tissues or cells in which the gene is expressed is identified.

The methodologies included above are meant to be examples only and do not exclude the use of any other technologies or experimental protocols familiar to anyone skilled in the art.

1. A method for identifying candidate genes capable of producing hybrid vigour in an animal or plant, comprising the steps of:
(i) comparing the nucleotide sequence of alleles of candidate genes isolated from an animal or plant which exhibits hybrid vigour with the nucleotide sequences from the corresponding alleles isolated from the parents of said animal or plant;
(ii) identifying nucleotide sequence differences in the alleles from said animal or plant which exhibits hybrid vigour which codes for amino acid sequence variation; and
(iii) identifying that the amino acid sequence variation between alleles of the candidate gene in said animal or plant is encoded by nucleotide sequences which are located within two or more different exons within the candidate gene.

2. The method according to claim 1, wherein the amino acid sequence variation is a conservative modified variation.

3. The method according to claim 1, wherein the amino acid sequence variation is a non-conservative modified variation.

4. The method according to claim 1, wherein the step of identifying the nucleotide sequence difference comprises the step of sequencing the nucleotide sequence isolated from said animal or plant.

5. The method according to claim 1, wherein the plant is selected from the group consisting of barley, rye, sorghum, maize, soybean, wheat, corn, potato, cotton, rice, oilseed rape (including canola), sunflower, alfalfa, sugarcane, banana, blackberry, blueberry, strawberry, raspberry, cantaloupe, carrot, cauliflower, coffee, cucumber, eggplant, grape, honeydew, lettuce, mango, melon, onion, papaya, pea, pepper, pineapple, spinach, squash, sweet corn, tobacco, tomato, watermelon, rosaceous fruits (including apple, peach, pear, cherry, and plum), vegetable brassicas (including broccoli, cabbage, cauliflower, brussel sprouts, and kohlrabi, currant, avocado, citrus fruits (including oranges, lemons, grapefruit, and tangerines), artichoke, cherries, nuts (including walnut and peanut), endive, leek, roots(including arrowroot, beet, cassava, turnip, radish, yam, sweet potato), and bean.

6. The method according to claim 1, wherein the animal is selected from the group consisting of a mammal and a fish.

7. The method according to claim 6, wherein the mammal is selected from the group consisting of the mammalian Orders Primates, Rodentia, Lagomorpha, Cetacea, Carnivora, Perissodactyla, and Artiodactyla.

8. The method according to claim 7, wherein the Artiodactyla is selected from the group consisting of the families Suidae, Tayassuidae, Hippopotamidae, Camelidae, Tragulidae, Giraffidae, Cervidae, Antilocapridae, and Bovidae.

9. The method according to claim 8, wherein the animal selected from Bovidae is an ungulate.

10. The method according to claim 9, wherein the ungulate is selected from the group consisting of cows, bulls, bison, buffalo, sheep, big-horn sheep, horses, ponies, donkeys, mule, deer, elk, caribou, goat, water buffalo, camels, llama, alpaca, and pigs.

11. The method according to claim 6, wherein the animal is a fish.

12. The method according to claim 11, wherein the fish is selected from the group consisting of zebrafish, European carp, salmon, mosquito fish, tench, lampreys, round gobies, tilapia, and trout.

13. The method according to claim 7, wherein the mammal is a human.

14. A method for identifying candidate genes capable of producing hybrid debility (HD) in an animal or plant, comprising the steps of:
(i) comparing the nucleotide sequence of alleles of candidate genes isolated from an animal or plant which exhibits said hybrid debility (HD) with the nucleotide sequences from the corresponding alleles isolated from the parents of said animal or plant;
(ii) identifying nucleotide sequence differences in the alleles from said animal or plant which exhibits said hybrid debility (HD) which codes for amino acid sequence variation; and
(iii) identifying that the amino acid sequence variation between alleles of the candidate gene in said animal or plant is encoded by nucleotide sequences which are located within two or more different exons within the candidate gene.

15. The method according to claim 14, wherein the amino acid sequence variation is a conservative modified variation.

16. The method according to claim 14, wherein the amino acid sequence variation is a non-conservative modified variation.

17. The method according to claim 14, wherein the step of identifying the nucleotide sequence difference comprises the step of sequencing the nucleotide sequence isolated from said animal or plant.

18. The method according to claim 14, wherein the plant is selected from the group consisting of a weed and other noxious plant.

19. The method according to claim 14, wherein the animal is a pest.

20. The method according to claim 19, wherein the pest animal is selected from the group consisting of a rodent, a rabbit, and a fish.

21. A method for producing hybrid vigour or hybrid debility in an animal or plant, comprising the steps of:
(i) comparing the nucleotide sequence of alleles isolated from a gene from an animal or plant which promotes hybrid vigour or hybrid debility with the nucleotide sequences from the corresponding alleles isolated from the parents of said animal or plant;
(ii) identifying nucleotide sequence differences in the alleles from said animal or plant which promote hybrid vigour or hybrid debility which code for amino acid sequence variation;
(iii) identifying that the amino acid sequence variation between alleles of the candidate gene in said animal or plant is encoded by nucleotide sequences which are located within two or more different exons within the candidate gene;
(iv) preparing a construct comprising nucleotide sequence from the alleles which promotes hybrid vigour or hybrid debility within said animal or plant;
(v) transforming said construct into a recipient plant-ef animal or plant cell; and
(vi) regenerating the animal or plant, which expresses said construct, from said cell.

22. A method for detecting the presence or absence of hybrid mRNA in a plant or animal comprising the steps of:
(i) isolating mRNA from a plant or animal;
(ii) comparing the nucleotide sequence of said mRNA to the corresponding coding sequences of the plant's or animal's alleles; and
(iii) determining whether or not the mRNA sequence comprises nucleotide sequences from two or more different exons.

23. A construct comprising a synthetic gene, the synthetic gene comprising exons from different alleles of a gene, wherein said exons code for amino acid sequence variation found in only one allele, such that said synthetic gene does not contain a nucleotide sequence that is the same as either allele and is capable of producing hybrid mRNA.

24. A method for using the construct according to claim 23 to overcome hybrid debility in a plant or animal and/or induce hybrid vigour in a plant or animal comprising the step of introducing said construct into said plant or animal.

25. The method according to claim 24, wherein the step of introducing said construct into said plant or animal is by transformation.

26. The method according to claim 25, wherein the step of transformation into the plant is selected from the group consisting of homologous recombination, microprojectile bombardment, PEG mediated transformation, electroporation, silicon carbide fibre mediated transformation, or Agrobacterium-mediated transformation.

27. A method for producing genetically engineered or transgenic non-human animal by inserting a synthetic gene into a non-human somatic cell or cell nucleus prior to transferring the somatic cell or cell nucleus, wherein said synthetic gene comprises exons from different alleles of a gene, wherein said alleles code for amino acid sequence variation, wherein the variation does not occur in the same allele.

28. A genetically engineered or transgenic non-human animal obtained by the method according to claim 27.

29. The method according to claim 27, wherein the cells are isolated from an animal selected from the group consisting of a mammal and a fish.
